(19) United States
(12) Patent Application Publication
(10) Pub. No.: US 2010/0056443 A1
(43) Pub. Date: Mar. 4, 2010

(54) RADIOPHARMACEUTICAL TO BE USED IN DISTINCTION BETWEEN INFLAMMATIONS AND INFECTIONS IN ORTHOPAEDIC IMPLANTS

(76) Inventors: Mauro Liberatore, L'Aquilla (IT); Alessandro Pala, Roma (IT)

Correspondence Address: NIXON & VANDERHYE, PC, 901 NORTH GLEBE ROAD, 11TH FLOOR, ARLINGTON, VA, 22203, US

(21) Appl. No.: 12/376,524

(22) PCT Filed: Aug. 1, 2007
(86) PCT No.: PCT/IB07/53029

§ 371 Date: Mar. 11, 2009

(30) Foreign Application Priority Data
Aug. 7, 2006 (IT) RM2006A000431

(51) Int. Cl. A61K 38/16; C07K 14/00; A61P 31/04

(52) U.S. Cl. 514/12; 530/380

(57) ABSTRACT

The present invention relates to a radio-pharmaceutical to be used in distinction between inflammations and infections in orthopaedic implants, containing one or more radiomarked human recombinant defensins of -α or -β type, in particular -β defensin of type 3.

The present invention relates to a radio-pharmaceutical to be used in distinction between inflammations and infections in orthopaedic implants, containing one or more radiomarked human recombinant defensins or defensins with identical primary structure obtained by synthetic route, of -α or -β type, in particular -β defensin of type 3.

### STATE OF ART

The orthopaedic implants are currently a valid and extremely commonly used means to restore the functionality of compromised joints or fractured scheletrical segments. In the United States of America only, over half million operations of hip or knee arthroprosthesis are currently performed every year. Considering this large population of patients with orthopaedic implants, the risk of implant infection, even if low in percentage (0.5-5%), must not be underestimated both for the number of patients which are likely to be subjected to such complication and for the infection's serious consequences. In the first two years as from implantation of the knee prostheses, the infection results to be the second cause for revising the arthoprosthesis after instability, if not even the first one. The debridement procedures with in situ maintenance of the prosthesis associated to the antibiotic therapy are not always able to eliminate the infection. With a certain frequency, the removal of the prosthesis with a subsequent new implant represent the only therapeutic option valid in order to eradicate the infection. Such drastic operations involve relevant phsycho-physical trauma for the patient subjected thereto, prolonged hospitalizations and considerable sanitary and social costs: the treatment of a single case of prosthetic infection has a cost greater than 50,000 dollars. Thereto it is to be added that, after the operation for revising the arthroprosthesis, there is a significantly high risk of infection relapse (up to 10%).

In clinical practice the infections secondary to orthopaedic implants (hip, knee and shoulder arthroprostheses, metallic synthesis of fractures, reconstruction of tendons, ligaments, etc.) are difficult to be diagnosed. In fact, the fundamental distinction between implant infection and not septic malfunction is often difficult. In fact, whereas the precocious post-operating prosthetic infections and the haematogenous ones are characterized by acute beginning and infection clinical signs, the prostheses' late infections show weaker inflammation signs, post-operation persistent chronic pain and/or precocious mobilization of the implant. For this reason, image diagnosis of infections assumes an important role even if, unfortunately, it is often difficult to be carried out considering the insufficient specificity of available diagnostic procedures. In fact, the computerized tomography (CT) and the nuclear magnetic resonance (NMR) suffer from the presence of metallic artifacts and the medico-nuclear methodologies, in particular the scintigraphic methodologies, even if characterized by greater diagnostic reliability, have a not insignificant incidence of false-positive cases, due to the unability of the commonly used radiopharmaceutical to distinguish between infection and inflammation.

Therefore, it is felt the need for having available a radiopharmaceutical to be used in a scintigraphic method reliable for the diagnosis and follow-up of the infections secondary to the orthopedic implants, able to distinguish between infections and inflammations.

Therefore, it is an object of the present invention a radiopharmaceutical to be used in the distinction between inflammations and infections in orthopaedic implants containing at least a radiomarked human recombinant defensin or a defensin with identical primary structure obtained by synthetic route of -α or -β type. Under human recombinant defensin, a defensin obtained with the DNA-recombinant technique is meant, whereas under defensin with identical primary structure obtained by synthetic route a defensin obtained by chemical synthesis from the sequence of aminoacids constituting the native peptide with or without disulfide bridges is meant.

Advantageously, according to the present invention, human recombinant defensins of β type, in particular of β-3 type, can be used.

### BRIEF DESCRIPTION OF THE DRAWINGS

Four tables are enclosed with the present invention which show:

FIG. 1 plates containing colonies of S. Aureus which have developed without adding radiomarked human recombinant HBD-3 at different dilutions of the sample (A=1:1345; B=1:13450) and plates (C and D) containing colonies of S. Aureus which have developed in presence of radiomarked HBD-3 at dilutions of the 'sample identical to the ones of A and B reported above;

FIG. 2 plates containing colonies of E. Coli which have developed without adding radiomarked human recombinant HBD-3 at different dilutions of the sample (A=1:1345; B=1:13450) and plates (E and F) containing colonies of E. Coli which have developed in presence of radiomarked HBD-3 (G and H) at dilutions of the sample identical to the ones of E and F reported above;

FIG. 3 shows the average run in time of the injected dose percentage of the same β-defensin-3-^{99m}Tc fixed per gram of normal tissue and of tissue wherein a lesion has been intentionally provoked in the rat;

FIG. 4 shows the run in time of the percentage of injected dose of human β-defensin-3 radiomarked with ^{99m}Tc per gram of tissue (in each one of the 9 examined rats in the normal tissue and in the experimentally induced lesions).

### DESCRIPTION OF THE INVENTION

Under radiopharmaceutical is meant a molecule of biological interest, or a cell conjugated to a radioisotope emitting ionizing radiations which, as a whole, has features such as to let foresee an “in vivo” use thereof in humans for diagnostic (planar scintigraphy, single-photon emission tomography, positron-emission tomography) or therapeutic purpose.

The radiopharmaceuticals according to the present invention can be used in distinction between inflammations and infections when the infections are caused by Gram-positive bacteria, for example Staphylococci, but also by Gram-negative bacteria, when such bacteria belong to the bacteria thereagainst the defensins act. In particular, among the Gram-positive bacteria, Staphylococci such as Staphylococcus Aureus included the multi-resistant strains, the strains of vancomycin-resistant Enterococcus faecium strains and Streptococci such as Streptococcus pyogenes can be cited; among the Gram-negative bacteria, Pseudomonas Aeruginosa and Escherichia coli can be mentioned.

Within the scope of the present invention, as orthopaedic implants, for example, hip, knee and shoulder arthroprostheses, metallic syntheses of fractures, reconstruction of tendons, ligaments and the like are meant.

The defensins used according to the present invention, both the human recombinant ones and those with identical primary structure obtained by synthetic route, are marked with known techniques with radionuclides which can be detected scintigraphically. Thereamong, as example the ^{99m}Tc can be cited. Obviously, within the scope of the present invention, other radionuclides and radioisotopes could be used.

Another object of the present invention is the use of at least a (human recombinant or synthetic) defensin of -α or -β type radiomarked for the production of a radiopharmaceutical for the distinction between inflammations and infections in orthopaedic implants.

Mammalian defensins are antimicrobial peptides constituted by 30-40 aminoacid residues, the molecular weight thereof is in the order of 4, 5-5 kD. In the defensin molecules, six cysteine residues are present that form three intramolecular disulfide bridges which stabilise a triple-stranded antiparallel β-sheet secondary structure. The mammalian defensins can be classified as α- or β-forms according to the spacing existing between their six cysteine residues and the configuration of the disulfide bridges. In solution, the α- or β-defensins have a similar three-dimensional structure. Both classes of defensins have been described in humans and rodents, whereas only α-defensins have been isolated in rabbits and Guinea pigs and only β-defensins in cattle, sheep and pigs. The α-defensins constitute 5-7% of the total proteins in neutrophils and they are present in high concentrations in the granules and phagocytic vacuoles existing within these cells. Generally, in mammals, α-defensins are present above all in the neutrophils and in the Paneth cells. Paneth cells are secretory cells containing many cytoplasmatic granules, located at the base of the crypts of Lieberkühn within the epithelium of the small intestine.

The β-defensins have been isolated from neutrophils, from other leukocytes, but also from epithelial cells in addition to plasma and urine.

Human α-defensins are arginine-rich peptides, constituted by 29-35 aminoacids. Their disulfide bridges connect cysteines 1-6, 2-4 and 3-5. Up to now, 5 different genes of the α-defensins and six molecules of human α-defensins have been described. The latter are the molecules HNP-1, HNP-3, HNP-4, HD-5 and HD-6. The peptide called HNP-2 is a truncated human α-defensin. HD-5 and HD-6 are expressed mainly in the Paneth cells. The current view is that these molecules are used to kill the microorganisms phagocytosed during the intracellular distribution to phagolysosomes. Furthermore, it is possible that a systemic distribution of human α-defensins by localization of neutrophils in the lung tissue and the secretion of such molecules from the granules of these cells may take place. HD-5 and HD-6 are expressed in the Paneth cells and in the granulocytes existing in the epithelium in the small intestine, therefore they are called enteric defensins.

Seat, kind of expression and activity of the human α-defensins are shown in Table I.

TABLE I
Classification of α-defensins
(Main function of the α-defensins: killing of the
phagocytosed microorganisms)

| | Expression | Expression | |
|---|---|---|---|
| Peptide | seat | type | Activity |
| HNP-1 | Inside | Constitutive | Bactericide against: |
| | neutrophils | | Gram-positive and |
| | | | Gram-negative bacteria |
| | | | at concentrations |
| | | | greater than 100 μl; |
| | | | Virus capsulates such |
| | | | as Herpes virus |
| HNP-2 | Inside | Constitutive | Like HPN-1 |
| | neutrophils | | |
| HNP-3 | Inside | Constitutive | Similar to HPN-1 |
| | neutrophils | | |
| HPN-4 | Inside | Constitutive | Similar to HpN-1 |
| | neutrophils | | |
| HD-5 | Paneth cells | Constitutive | Bactericide against: |
| | of crypts of | | Escherichia Coli, |
| | Lieberkühn, | | Lysteria |
| | epithelial | | monocytogenes, |
| | cells | | Salmonella |
| | of the feminine | | typhymurium, C. albicans |
| | reproductive | | |
| | apparatus, | | |
| | placenta and | | |
| | fetal membranes | | |
| HD-6 | Like HD-5 | Constitutive | Similar to HD-5 |

Human β-defensins, too, contain 35-45 aminoacid residues, thereamong six cysteines, and they are characterized by a different spacing pattern of disulfide bridges with respect to the α-defensins. The β-defensins are mainly expressed in epithelial tissues, which constitute the first line of defense between the organism and the environment. The first human β-defensins HBD-1 and HBD-2 were described in 1995 and 1997, respectively. The transformations, the intra-cellular deposits and the mechanism for releasing β-defensins are not yet wholly defined. Seat, kind of expression and activities of human β-defensins are shown in Table II.

TABLE II
Classification of β-defensins
(Main function of β-defensins: contributing to the first
line of defense between the organism and the environment)

| | Expression | Expression | |
|---|---|---|---|
| Peptide | seat | type | Activity |
| HBD-1 | Cheratinocytes, | Constitutive | Bactericide* |
| | human | | against: |
| | epithelial | | Escherichia |
| | derived from | | Coli at micro- |
| | trachea, | | molecular concentrations; |
| | bronchi, | | Other Gram- |
| | small | | negative bacteria |
| | airways, | | at concentrations |
| | mammary glands, | | between 60 and |
| | parotide, | | 500 μg/ml |
| | buccal mucosa, | | |
| | tongue, gums, | | |
| | small intestine, | | |
| | pancreas, | | |
| | kidney, prostate, | | |
| | testicle, vagine, | | |
| | uterus, Falloppian | | |
| | tubes, placenta | | |
| | and thymus | | |
| HBD-2 | Cheratinocytes, | Induced | Bactericide |
| | gum, | (by IL-1β | against Gram- |
| | tracheal | TFN, | negative bacteria |
| | epithelium | bacterial | and yeasts |
| | | lipopoly- | Bacteriostatic |
| | | saccharide | against S. Aureus |
| | | | only at concentrations |
| | | | of |
| | | | 100 μg/ml or |
| | | | greater |
| HBD-3 | Strong | Induced | Bactericide |
| | expression | (by TFN-α | against: |
| | in cheratinocytes | γ interferon | Gram-negative |
| | and in | and contact | bacteria (p. Aeruginosa, |
| | the tonsillar | with bacteria | E. Coli) |
| | tissue; | inactivated | Yeasts |
| | low expression | by heat) | Gram-positive |
| | in the respiratory, | | bacteria\hat{ } |
| | gastro- | | (Streptococcus |
| | intestinal and | | pyogenes, S. Aureus) |
| | genitourinary | | |
| | epithelium. | | |
| | Nonepithelial | | |
| | tissues (leucocytes, | | |
| | heart and skeletal | | |
| | muscles) | | |
| HBD-4 | Testicole, | Constitutive | Weak antimicrobial |
| | gastric antrum | | activity |
| | and neutrophils; | | against: |
| | Ephitelia of: | | E. Coli, S. cerevisiae, |
| | thyroid, | | S. aureus, |
| | lung, uterus, | | S. pneumoniae, |
| | kidney | | B. cepacea |
| | | | (MIC > 100 μg/ml); |
| | | | strong |
| | | | antimicrobial |
| | | | activity against: |
| | | | S. Carnosous |
| | | | (MIC = 4.5 μg/ml) |
| | | | and P. aeruginosa |
| | | | (MIC = 4.1 μg/ml) |

*The antimicrobial activity is reduced in presence of high concentrations of sodium chloride in the airways.
\hat{ }Including the strains of multi-resistant S. aureus and vancomycin-resistant Enterococcus Faecium.

Human recombinant beta-defensin-3 (HBD-3) is a antimicrobial peptide weighing 5,15 kDa discovered independently by the groups of Harder, Garcia and Jia in 2001.

Similar to other human beta-defensins (HBD-1) and (HBD-2), HBD-3 has bactericide activity against Gram-negative bacteria (P. aeruginosa, E. coli) and the yeast C. albicans. Furthermore, HBD-3 is bactericide against Gram-positive bacteria such as Streptococcus pyogenes and S. aureus (including the multi-resistant strains of S. aureus) and also against the vancomycin-resistant Enterococcus faecium. Therefore, HBD-3 fills the gap existing in the activity spectra of HBD-1 and HBD-2 concerning the Gram-positive bacteria.

It has been demonstrated, and also this forms part of the present invention, that HBD-3, obtained by chemical synthesis from the aminoacid sequence constituting the native peptide, keeps its own biological activity, independently from the position of disulfide bridges (equal or different from the native peptide) and also in absence of these bonds (absence of cysteine).

From this point of view, HBD-3 marked with ^{99m}Tc could be an interesting radiopharmaceutical, considering that it binds selectively to the bacterial membrane and, in particular, to the membrane of S. Aureus. In fact, it is well known that great part of infections secondary to orthopaedic implants are due to Gram-positive aerobic bacteria, above all Staphilococci (S. Aureus 34%, S. epidermidis 32%).

The first results obtained in animals with this molecule (compare section “Detailed description of the invention”) show that the captation ratio between the infective lesion and the nonseptic inflammatory lesion is significantly high and, furthermore, that there are no significant concentration differences in the marked product between the healthy tissues and the inflammatory lesion, after at least 3 hours have elapsed as from the injection of the product itself.

An additional object of the present invention is a kit of analyses for the distinction between inflammations and infections in orthopaedic implants comprising:

a. at least a human recombinant defensin;

b. a radioisotope able to bind to the defensin of stage a.

c. a pH-adjusting substance

d. means for measuring pH and

e. a solvent.

In the present description, as radioisotope the ^{99m}Tc will be referred to, but also other radioisotopes could be used.

The necessary laboratory equipment also includes devices for manipulating and transferring the used substances and means for eliminating the unwished products.

As biologically active product, human recombinant β-defensins of type 3 (HBD3) are preferred; said defensins lie among the materials needed under lyophilized form. As far as the radioisotope to mark the defensin is concerned, the anion ^{99m}Tco₄₋ to be reduced to cation at lower oxidation state is preferred. Examples of reducing agents are Na boranocarbonate which generates a complex of the monovalent cation technetium, exa-coordinated with H₂O and CO [^{99m}Tc(CO)₃(H₂O)₃)]⁺; sodium hydrosulphite; (stannous) salts of Sn⁺² such as chloride; K borohydride (KBH₄).

With compounds belonging to the last three mentioned classes, reduced ^{99m}Tc can be obtained which can be subsequently complexed by chelating agents such as the diethylen-triamine-pentacetic acid (DTPA) able to bind it to molecules of interest. The reducing agent is always present in strong excess, considering the high specific radioactivity of ^{99m} Tc. The ^{99m}Tc is added into the same vial containing the reducing agent.

As pH-adjusting means, an acqueous solution of HCl has been used. The detection will take place then preferably with scintigraphic methodologies.

An example of methodological application of the invention is the following:

1. A 10-ml vial which can be sealed hermetically, but with a rubber plug, which can be bored, wherein the marking reaction takes place, containing a reducing agent for the ^{99m}Tc₄⁻ in suitable quantities;

2. A flask containing β-defensin-3 (HBD3);

3. A flask containing HCl 0.2 5 N necessary to bring the pH of the marked product back to a value of 7.8-8.0;

4. A pH indicator paper;

5. 3 sterile 2-ml syringes for manipulating and transferring the various solutions.

6. An ultrafilter for centrifuge of “Centricon” type (Amicon) or, alternatively, a column for gel-filtration of PD-10 type (General Electric Healthcare).

### Embodiment Examples of the Invention

A typical marking experiment involved:

the use of the reduction system based upon the use of a mixture of Na Boranocarbonate (4.5 mg) with Na Tartrate (8.5 mg), Na Tetraborate (2.5 mg) and Na Carbonate (7.15 mg) and the reconstruction of the lyophilizate with 1 ml of ^{99m}Tc₄⁻. The hermetically sealed vial was placed in boiling water bath for 20 minutes, a sufficient time to reduce the ^{99m}Tc₄⁻ and to form the complex thereof with the carbon monoxide generated “in situ” and with water.

1. The solution was brought back to room temperature and then brought to pH 7.8-8.0 with a solution of HCl 0.25 N, added drop by drop, with the help of a pH indicator paper.

2. The β-defensin-3 (100 μl of sterile and apyrogen H₂O) was added to the solution and it was left to react for about 1 hour.

3. The ^{99m}Tc− β-defensin-3 was released from the reagents and from the reaction products through gel-filtration onto Sephadex G-25 (column of about 15 ml of total volume) equilibrated and eluted in 1-ml fractions with physiological saline. The first peak, eluted in the fractions 3-6, contained about 4 mCi of ^{99m}Tc, determined by counting suitable dilutions of the cromatographic fractions.

HBD-3 Marking

A 100-μg aliquote of recombinant HBD-3 was marked with ^{99m}Tc⁺ exa-coordinated with 3 molecules of CO and H₂O. The boronocarbonate was diluted with 1 mL of distilled water containing about 1850 MBq (50 mCi) of ^{99m}Tc and boiled for 20 minutes before bringing the pH to 8.0 by adding HCl in solution. 300 μl of H₂O containing recombinant HBD-3 were then added to ^{99,}Tc0 and the mixture was left to react for one hour at room temperature. The marked peptide was subsequently separated from the not reacted ^{99m}Tc by means of gel-filtration onto a single-use column of Sephadex G-25 (PD-10). Aliquots of the solution eluted from the column were counted in a well-like counter.

The specific activity of the radiopharmaceutical is comprised between 250 and 500 KBq/μg and the average concentration of defensin between 5 and 10 μg/ml.

“In vitro” Control of the Biological Activity of the Radiomarked HBD-3

The control was performed as described by Harwing et al. Methods Enzymology 1994, 236: 160-172. In particular, the aliquote portion (20 μl) of a bacterial suspension of S. Aureus containing 5×10⁶ CFU/ml was incubated (2 hours at 37° C.) with 200 μl of radiomarked HBD-3 (1.2 μg) and 75 μl of a suitable dilution of a culture broth (Tryptic Soy Broth). After incubation, two samples with dilution factors equalling to 1:1345 and 1:13450 were derived from the mixture. Twentyfive μl of these samples were collected and sown with sterile rod onto plates containing a culture medium (agar plate). After incubation for 24 hours at 37° C. the colonies were counted. A value higher than 800 CFU was considered not assessable due to the difficulty in counting.

An analogous experiment was carried out, under the same conditions, by using 200 μl of sterile buffer instead of the radiomarked HBD-3.

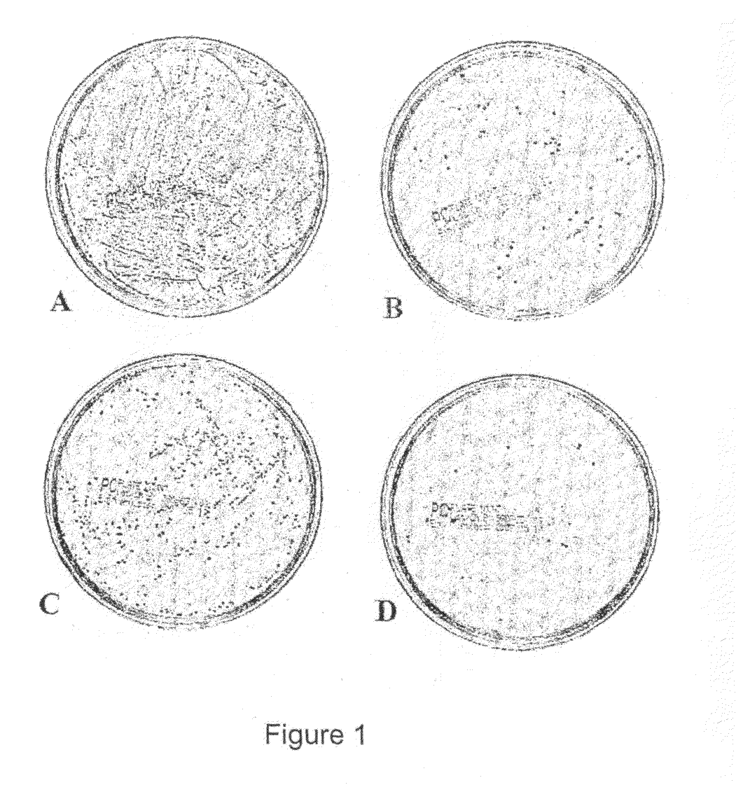

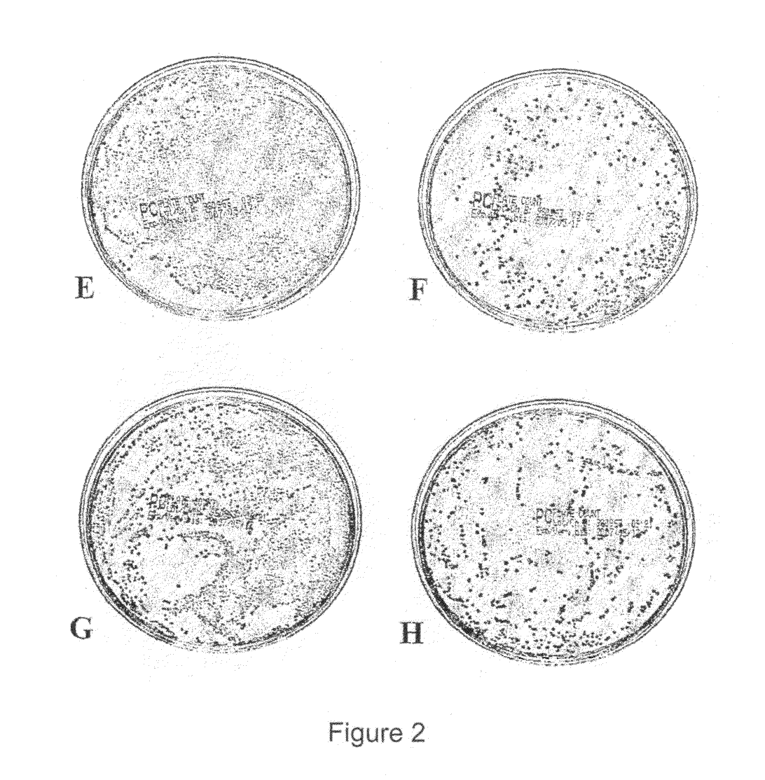

Both tests illustrated above, aimed at testing the sensibility of S. Aureus to the radiomarked HBD-3, were repeated, under identical conditions, with E. Coli with the same purposes. The results are shown in Table III and in FIGS. 1 and 2.

TABLE III
Comparison between the development of
colonies without and after adding radiomarked HBD-3

| Number of CFU existing in the plates | |
|---|---|
| S. Aureus | E. Coli |

| | Dilution | Dilution | Dilution | Dilution |
|---|---|---|---|---|
| | 1:1345 | 1:13450 | 1:1345 | 1:13450 |
| Without | >800 | 59 | >800 | 400 |
| radiomarked | | | | |
| HBD-3 | | | | |
| In presence | 348 | 17 | >800 | 310 |
| of radio- | | | | |
| marked HBD-3 | | | | |

The results have been interpreted as indicating a maintained biological activity of HBD-3 after marking with ^{99m}Tc, even if such activity results to be greater towards S. Aureus with respect to what happens towards E. Coli.

Infection Detection by Means of ^{99m}Tc-HBD-3 in an Experimental Animal Model

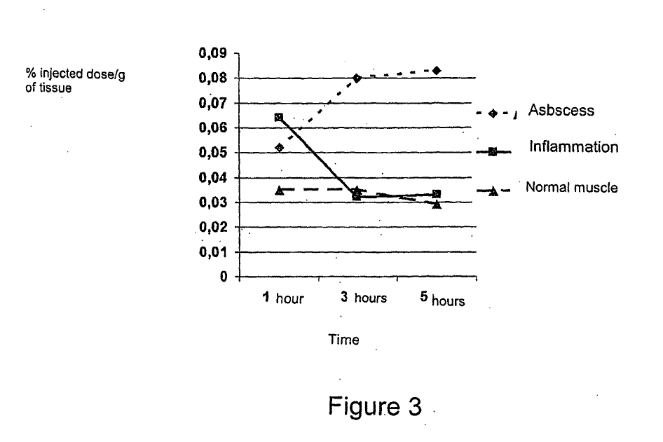

Various doses of radiomarked HBD-3 (from 1,6 to 5,2 μg for each chosen period of time) were injected in three groups of Wistar male rats wherein an infection by S. Aureus and a sterile inflammation by carragenate had been experimentally induced. Tissue samples were collected from the infection and inflammation seats after 1, 3 and 5 hours as from the injection of the tracer for each group of rats. Such samples were counted in a well-like counter. The countings expressed in terms of cpm/g of tissue were compared to the countings of properly collected normal muscle tissue samples. The tracer's captation in the infection seats, after 5 hours as from the tracer's injection, resulted to be 3 times higher than that found in the inflammation seats (FIG. 3), whereas in the samples collected after one hour as from the injection there were no significant differences between the infection and inflammation seats. Furthermore, no significant fixation differences of tracer between the inflammatory lesions and the normal muscles were detected, in the whole experiment. The results have been interpreted as indicating a specific bond of radiomarked recombinant HBD-3 with the S. Aureus.

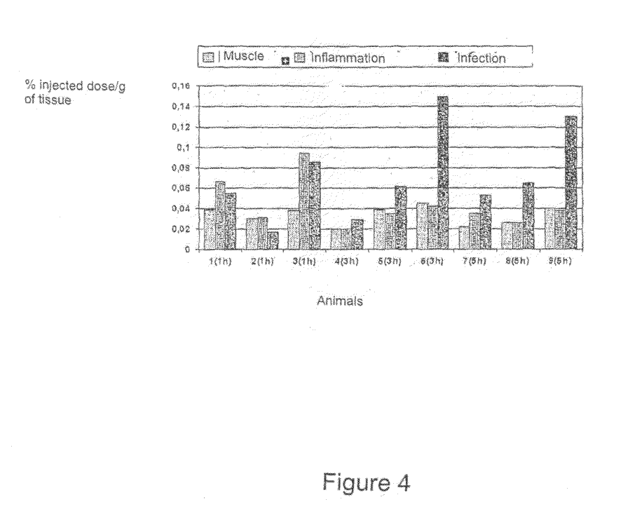

FIG. 4 shows such results expressed by single studied animal. The rats Nr. 3, 6 and 9 had received a weight dose in marked product higher than the rats Nr. 2, 5 and 8 which, in turn, had received a dose of the same product higher than the rats 1, 4 and 7. As it can be seen, a greater ratio between countings measured onto the abscess seat and those recorded onto the inflammation seat and onto the normal muscle corresponds to a higher dose of radiomarked peptide. Such run lets assuming a dose-depending ratio of dependency of the radiopharmaceutical captation.

1. Radiopharmaceutical to be used in distinguishing between inflammations and infections in orthopaedic implants containing at least a radiolabeled defensin of -β type.

2. Radiopharmaceutical according to claim 1, wherein said at least one defensin is a human recombinant defensin or a defensin with identical primary structure obtained by synthetic route.

3. Radiopharmaceutical according to claim 1, wherein said at least one defensin is of β-3 type.

4. Radiopharmaceutical according to claim 1, wherein said infections in said implants are provoked by Gram-positive and/or Gram-negative bacteria.

5. Radiopharmaceutical according to claim 1, wherein said infections are induced by Gram-positive bacteria such as Staphylococcus auerus including the multi-resistant strains and the vancomycin-resistant strains of Enterococcus faecium, Streptococci such as Streptococcus pyogenes and by Gram-negative bacteria such as Pseudomonas aeruginosa Escherichia coli and the yeast Candida albicans.

6. Radiopharmaceutical according to claim 1, wherein said orthopaedic implants are selected from the group consisting of hip, knee and shoulder arthroprostheses, metallic synthesis of fractures, reconstruction of tendons, ligaments and the like.

7. Radiopharmaceutical according to claim 1, wherein said at least one defensin is labeled with ^{99m}Tc.

8. Use of at least a radiomarked defensin of -β type for producing a radiopharmaceutical for the distinction between inflammations and infections in orthopaedic implants.

9. Use according to claim 8, wherein said at least one defensin is a human recombinant defensin or a defensin with identical primary structure obtained by synthetic route.

10. Use according to claim 8, wherein said at least one defensin is of β-3 type.

11. Use according to claim 8, wherein said infections in said implants are induced by Gram positive and/or Gram negative bacteria.

12. Use according to claim 8, wherein said infections are provoked by Gram-positive bacteria such as Staphylococcus auerus including the multi-resistant strains and the vancomycin-resistant strains of Enterococcus faecium, Streptococci, such as Streptococcus pyogenes and by Gram-negative bacteria such as Pseudomonas aeruginosa Escherichia coli and the yeast Candida albicans.

13. Use according to claim 8, wherein said orthopaedic implants are selected from the group consisting of hip, knee and shoulder arthroprostheses, metallic synthesis of fractures, reconstruction of tendons, ligaments and the like.

14. Use according to claim 8, wherein said at least one defensin is labeled with ^{99m}Tc.

15. Kit for analyses for determining the difference between inflammation and infection in orthopaedic implants including:
a) at least a defensin of -β type;
b) a radioisotope able to bind to the defensin of stage a;
c) a pH-adjusting substance d) means for measuring pH and
e) a solvent.

16. Kit according to claim 15, wherein said at least one defensin is a human recombinant defensin or a defensin with identical primary structure obtained by synthetic route.

17. Kit according to claim 15, further containing devices for manipulating and transferring the used substances and means for eliminating the unwished products.

18. Kit according to claim 15, wherein said at least one β-defensin is of β-3 type.

19. Kit according to claim 15, wherein said radioisotope is ^{99m}Tc.
